(19) United States
(12) Patent Application Publication
(10) Pub. No.: US 2018/0177700 A1
(43) Pub. Date: Jun. 28, 2018

(54) HAIR REMOVAL COMPOSITIONS AND METHODS

(71) Applicant: ADAM M. ROTUNDA, IRVINE, CA (US)

(72) Inventor: ADAM M. ROTUNDA, IRVINE, CA (US)

(21) Appl. No.: 15/392,944

(22) Filed: Dec. 28, 2016

(51) Int. Cl. A61K 8/49; A61Q 7/02; A61Q 9/00; A61K 8/46

(52) U.S. Cl. CPC A61K 8/4973; A61Q 7/02; A61K 2800/74; A61K 8/466; A61Q 9/00

(57) ABSTRACT

Compositions and methods for selectively and permanently removing hair from the body. The compositions are formulated to be topically applied upon discrete areas of the body and contain an agent present in an amount sufficient to cause permanent alopecia about the area upon which the composition is applied. Exemplary of such agents include taxanes, such as docetaxel and paclitaxel, and busulfan and other agents known in the art to cause permanent chemotherapy-induced alopecia (CIA). The compositions and methods of applying the same are limited exclusively to transdermal application and operative to induce a localized CIA, and thus avoid systemic distribution of the CIA agent throughout the body. The compositions may further be deployed through known transdermal application mechanisms, such as transdermal patches and the like.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

The present invention pertains to the field of permanent hair removal using a topically applied pharmaceutical composition operative to reach the hair papilla and inactivate it via localized chemotherapy-induced non-scarring alopecia, therefore resulting in permanent inactivation of the follicle and, consequently, permanent non-scarring hair loss (otherwise known as non-scarring alopecia). By definition, ‘permanent’ hair loss is absence of or incomplete growth (i.e., not returning to baseline hair growth, including thin, patchy, lighter or sparse growth) hair growth at 6 months or after drug exposure.

Hair is widely distributed in the skin and is believed to serve a number of biological or physiological purposes. These include thermal protection, as well as protection from abrasion and from sun exposure. However, such functions have largely been lost in humans, on whom hair is usually kept or removed from various parts of the body for cosmetic reasons. In this regard, in females body hair is typically seen as unpleasant and its presence can cause distress in affected women, who generally prefer smooth bodies. Likewise, in males the presence of excessive body hair or its localization in particular areas (e.g., the back, shoulders) is unwanted and there is a growing market pressure for methods to remove them.

Current hair removal methods can be classified in two categories: 1) methods which target the hair shaft (e.g., shaving, tweezing, waxing, sugaring) and 2) methods which try to target the hair dermal papilla (e.g., electrolysis and laser treatment). The former methods are easy to implement but can only provide temporary effect with the hair eventually growing back, sometimes within a day or two. The latter, targeting the dermal papilla, can advantageously effectuate permanent hair loss; however, such approach is far more complicated due to the fact the dermal papilla lies deep in the dermis and cannot be removed by mechanical means. As a result, difficulties arise in trying to selectively attack the dermal papilla without causing skin damage, such as scarring or burning, at the same time.

With respect to the most widespread and well-known of the permanent hair removal techniques, namely, electrolysis and laser treatment, each have significant drawbacks. Hair electrolysis involves inserting a fine metal conductor down into the hair shaft through which an electric current is applied and the hair bulb subsequently destroyed either by overheating (thermal electrolysis) or by the electrochemical local generation of caustic compounds (galvanic electrolysis). Because the hair bulb is targeted, the technique provides permanent hair removal. The main drawbacks of electrolysis are its slow rate (only one follicle can be treated per application), its painfulness, and the risk of scarring if sufficient care is not exerted. Nevertheless, electrolysis is now very popular throughout the world.

Laser hair removal, currently the most modern method available for permanent hair removal, works by applying intense pulses of laser light at a certain wavelength on the hair after it has been shaven off. Insofar as the hair shaft has a different, darker color than the rest of the skin, it will absorb more of the laser radiation. This results in the overheating of the shaft itself, which in turn causes the transmission of heat on to the dermal papilla, with the ultimate objective of permanently damaging the papilla.

While effective, laser hair removal is not perfect and it does reduce the coarseness of most hair. Moreover, because of its principle of action, laser hair removal works best where there is a strong color contrast between hair and surrounding skin (i.e. ideally dark hair on white skin). When this is not the case, such as for light hair or dark complexions, the method is far less effective and can cause skin damage, and instead often cause irritation. Such methodology is also expensive because of the equipment involved and must be carried out in specialized salons. Finally, because of its unpredictable efficiency, it cannot be considered a “permanent hair removal” but a “permanent hair reduction” method and it is so advertised.

New technologies, such as pre-application of topical “photo-particle” based destruction of the follicular unit, may offer patients a potentially more efficient and effective treatment than laser hair removal alone (and thus allow for lighter hair to be treated); however, these methods still rely on selective destruction of the hair follicle with a specialized device and their effectiveness and safety are as of yet unknown.

An FDA approved topical medication, VANIQA® (eflornithine hydrochloride) Cream, 13.9%, produced by Allergan, is a prescription medication applied to the skin for the reduction of unwanted facial hair in women. While there are no studies examining the inhibition of the enzyme ornithine decarboxylase (ODC) in human skin following the application of topical eflornithine, there are studies in the literature that report the inhibition of ODC activity in skin following oral eflornithine. Notwithstanding, the drug is approved only for use to inhibit facial and chin hair. In a study of over 594 patients using topical eflornithine hydrochloride versus placebo for 24 weeks in the pivotal trial, 5% (vs 0% in placebo), 27% (vs 8% in placebo), and 26% (vs 26% in placebo) had “Clear, Marked” or “Improved” physician reported changes in unwanted facial hair. However, the results were transient, result in transient inhibition of hair growth, and returned to baseline eight weeks after the drug application.

Because there are few choices as to non-invasive methods for permanent hair removal and given the drawbacks associated with presently available techniques, permanent hair removal products are needed that are capable of being easily and readily applied to the skin and can effectively reduce the regrowth of hair to provide permanent hair removal.

### BRIEF SUMMARY

The present invention specifically addresses and alleviates the above-identified deficiencies in the art. In this regard, the present invention is directed to compositions and methods for selectively and permanently removing hair from the body via chemotherapy-induced alopecia (CIA). To that end, the compositions contain an effective amount of an agent operative to cause permanent CIA that is transdermally applied upon discrete portions of the body where permanent hair loss is desired. The compositions of the present invention and the manner by which they are applied are specifically designed and formulated for localized application that subsequently produces a localized CIA, and thus avoid any type of systemic distribution of the CIA agent that could cause generalized CIA, which the present invention expressly seeks to avoid.

With respect to the agents for use in inducing permanent CIA per the present invention, it is believed that any of a variety of known agents operative to cause permanent CIA may be utilized. Among the agents believed to be best suited for the practice of the present invention include: taxanes, including docetaxel and paclitaxel, busulfan, and other related compositions. Other agents known to cause permanent CIA may also be utilized in the practice of the present invention, and can include the disulfan, cyclophosphamide, thiotepa, tepa, melphalan, etoposide, carboplatin, cisplatin and ifosfamide, doxorubicin, dactinomycin, vincristine among others known in the art. All such aspects are well-known in the art, have been extensively utilized as anti-cancer therapies, and can be readily formulated and applied to produce the desired localized CIA effect.

Along these lines, it is likewise contemplated that a combination of agents may be utilized to produce a permanent CIA, as may be desired for a particular application. For example, a combination of carboplatin may be used in combination with cyclophosphamide as it is known in the art that such combination produces a substantially increased incidence of hair loss. For purposes of the present invention, it is believed that taxanes and busulfan are a preferred CIA agent as such class of agents induce nearly 100% hair loss, as well as hair loss of all different types of body hair, including scalp and facial (eyebrows, eyelashes, moustache, beard), axillary (underarm), pubic (“bikini”), and leg hair.

For any particular agent deployed per the teachings of the present invention, such agent will be formulated to be applied on the surface of the skin where permanent hair removal is desired. To that end, the agent is applied via a pharmaceutical composition in an amount sufficient to produce permanent CIA at the specific location where applied in order to obtain a local effect. In order to formulate a specific pharmaceutical composition operative to effectively administer the CIA agent in amounts sufficient to achieve the localized hair loss, such pharmaceutical compositions may be formulated per any of a variety of known techniques that enable such CIA agents to be topically deployed. Accordingly, additional inactive ingredients, such as citric acid, polyethylene glycol, propylene glycol, stearyl alcohol, cocoa butter, cotton seed oil, olive oil, white petrolatum, water, mineral oil, methyl paraben, propyl paraben and other known base materials that can be mixed and formulated as lotions, creams, ointments and the like may be utilized in the formulation of such pharmaceutical compositions.

As will be readily understood by those skilled in the art, due to potentially profound differences by which the variety of CIA agents as used in the present invention may be absorbed through the skin via transdermal drug delivery, in those cases when a particular agent is not readily absorbed through the skin, other methodologies and/or compounds may be used in order to enhance the rate of absorption so as to cause more of the agent to penetrate the skin in a given unit of time. Accordingly, it is understood that the compositions of the present invention may deploy additional components which act as an absorption promoter to cause the CIA agent to more readily permeate through the skin. Additionally, it is contemplated that other techniques, such as lightly abrading the skin, photodynamic therapy (PDT), needling (ie, microneedling or needle rolling), lasering and/or other mechanical means of disrupting the top most (epidermal) skin layer, and/or applying electrical current to the skin so as to facilitate the ability of a particular agent to penetrate into the skin in an amount sufficient to produce a permanent CIA about the desired localized area.

In the case of taxanes, and in particular docetaxel, it is well-known that such drug is a highly hydrophobic drug that needs solvents to enhance its solubility and enable its formulation. In order to produce the desired permanent CIA, it is contemplated that the docetaxel must be applied in amounts ranging from 20 to 100,000 mg/square decimeter of skin, and in a more refined embodiment applied in amounts from 200 to 10,000 mg/square decimeter of skin, and in a most highly preferred embodiment, it is administered in an amount ranging from 200 to 2000 mg of docetaxel per decimeter of skin to be treated. According to one preferred formulation, it is contemplated that docetaxel may be applied via a pharmaceutical composition wherein the docetaxel component will be present in the composition in an amount ranging from 0.2 to 10 percent by weight, and in a more highly preferred embodiment from 0.2 to 2 percent by weight, whereby the remaining ingredients will comprise inactive materials. The composition will also preferably be formed as a cream, lotion, ointment, gel, foam, wax, spray or other formulation for ease of topical application. Such compositions may be applied as a single, one-time application or, alternatively, may be applied according to a regimen ranging from 1-3 times per day for up to 365 days.

In the case of busulfan, it is contemplated that the busulfan will be applied in amounts ranging from 20 to 100000 mg/square decimeter of skin, and in a more refined embodiment applied in amounts from 20 to 10000 mg/square decimeter of skin. In a most highly preferred embodiment, busulfan is administered in an amount ranging from 2 to 2000 mgs of busulfan per decimeter of skin to be treated. According to one preferred formulation, it is contemplated that busulfan may be applied via a pharmaceutical composition, wherein the busulfan component will be present in the composition in an amount ranging from 0.02 to 10 percent by weight, and in a more highly preferred embodiment 0.2 to 2 percent by weight, with the remaining ingredients comprising inactive materials. The composition will also preferably be formed as a cream, lotion, ointment, gel, foam, wax, spray or other formulation for ease of topical application. Such compositions may be applied as a single, one-time application or, alternatively, may be applied according to a regimen ranging from 1-3 times per day for up to 365 days.

Advantageously, the compositions of the present invention will be formulated so as to be applied directly onto the skin by manual application. Alternatively, to the extent desired, the compositions may be deployed through a transdermal drug delivery system, such as a transdermal patch using transdermal delivery mechanisms well-known in the art.

Upon completion of a particularly prescribed application regimen, the CIA agents will have thus been administered in amounts sufficient to cause permanent CIA. To the extent a sub-optimal amount of the CIA agent was applied, the application regimen may be modified such that greater amounts are applied, more frequently applied and/or applied in a longer duration so as to ultimately produce the desired permanent CIA.

### DETAILED DESCRIPTION

The detailed description set forth below is intended as a description of the presently preferred embodiment of the invention, and is not intended to represent the only form in which the present invention may be implemented or performed. The description sets forth the functions and sequences of steps for practicing the invention. It is to be understood, however, that the same or equivalent functions and sequences may be accomplished by different embodiments and that they are also intended to be encompassed within the scope of the invention.

The present invention is directed to compositions and methods for the permanent removal of hair by chemotherapy-induced alopecia (CIA). The compositions comprise an effective amount of an agent known to cause permanent non-scarring CIA that is formulated to be topically applied and transdermally delivered on selectively chosen, discrete areas of the body. In a preferred embodiment, discussed more fully below, the composition may be applied manually or through a conventional transdermal drug delivery mechanism, such as a transdermal patch and the like. In this regard, the compositions of the present invention, as well as the manner by which the same are applied, are specifically formulated for localized application on discrete areas of the body where hair removal is desired, and are specifically formulated and intended to be implied in a manner that does not involve any systemic distribution of the CIA agent. Rather, the objectives of the present invention are to provide a composition and a manner of applying the same that is exceedingly simple to administer and provide exceptionally effective, permanent hair loss on only those portions of the body as selectively chosen by the individual user.

Bearing such principles in mind, it is contemplated that the agent operative to induce the permanent non-scarring CIA effect may take any of those agents known in the art or later discovered that impart permanent CIA effects. Among the most well-known of such agents include anti-cancer chemotherapy agents, and in particular the class of chemotherapy agents known as taxanes and busulfan, which are well-understood an extensively utilized to treat cancer. In this regard, taxanes are drugs known as mitotic inhibitor and a type of antimicrotubule agent. Perhaps the most well-known of the taxanes are paclitaxel, whose chemical structure is shown below,

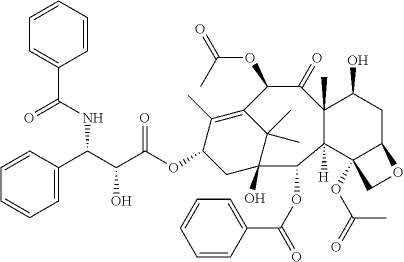 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

and docetaxel, whose molecular structure is shown below.

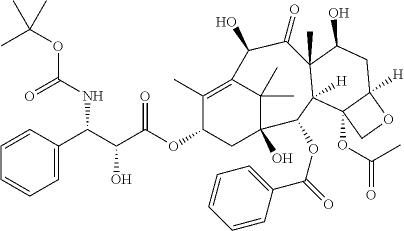

With regard to busulfan, it is a well-understood an extensively utilized to treat cancer. Busulfan is known is an alkylsulfonate, an alkylating agent that forms DNA-DNA intrastrand crosslinks between the DNA bases guanine and adenine and between guanine and guanine. Busulfan's molecular structure is shown below,

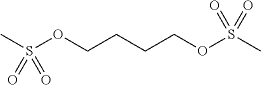

The medical use, mechanism of action and side effects associated with taxanes, and in particular paclitaxel and docetaxel referenced above, as well as busulfan, are extremely well-known in the art. Such agents are believed to be exceptionally effective in the practice of the present invention due to the ability of such compositions to cause CIA in virtually all types of individuals to which such compositions are administered. Moreover, such CIA agents are operative to remove all types of hair anywhere about the body. Accordingly, it is believed that when specifically applied to a discrete area to which permanent hair removal is desired, taxanes and busulfan represent a preferred agent for effectuating selective CIA.

In the case of taxanes, and in particular docetaxel, it is also well-known that such drug is a highly hydrophobic drug that needs solvents to enhance its solubility and enable its formulation. In order to produce the desired permanent CIA, it is contemplated that the docetaxel must be applied in amounts ranging from 20 to 100,000 mg/square decimeter of skin, and in a more refined embodiment applied in amounts from 200 to 10,000 mg/square decimeter of skin. In a most highly preferred embodiment, it is administered in an amount ranging from 200 to 2000 mgs of docetaxel per decimeter of skin to be treated. According to one preferred formulation, it is contemplated that docetaxel may be applied via a pharmaceutical composition wherein the docetaxel component will be present in the composition in an amount ranging from 0.2 to 10 percent by weight, and in a more highly preferred embodiment 0.2 to 2 percent by weight. The composition will also preferably be formed as a cream, lotion, ointment, gel, foam, wax, spray or other formulation for ease of topical application. Such compositions may be applied as a single, one-time application or, alternatively, may be applied according to a regimen ranging from 1-3 times per day for up to 365 days.

In the case of busulfan, it is contemplated that the busulfan must be applied in amounts ranging from 20 to 100000 mg/square decimeter of skin, and in a more refined embodiment applied in amounts from 20 to 10000 mg/square decimeter of skin and in, and in a most highly preferred embodiment, it is administered in an amount ranging from 2 to 2000 mgs of busulfan per decimeter of skin to be treated. According to one preferred formulation, it is contemplated that busulfan may be applied via a pharmaceutical composition comprising the ingredients below and their relative amount by weight: In a preferred embodiment, the busulfan component will be present in the composition in an amount ranging from 0.02 to 10 percent by weight, and in a more highly preferred embodiment 0.2 to 2 percent by weight. The composition will also preferably be formed as a cream, lotion, ointment, gel, foam, wax, spray or other formulation for ease of topical application. Such compositions may be applied as a single, one-time application or, alternatively, may be applied according to a regimen ranging from 1-3 times per day for up to 365 days.

The formulation and manufacture of the compositions disclosed herein would be readily understood and within the skill level of the ordinary artisan. Indeed, it is contemplated that formulation of pharmaceutically-stabile compositions for use in deploying taxane-based or busulfan CIA agents may take any of a variety of existing forms known in the art, exemplary of which include those disclosed in Published PCT Application No. PCT/US2005/019017, entitled A MIXTURE FOR TRANSDERMAL DELIVERY OF LOW AND HIGH MOLECULAR WEIGHT COMPOUNDS, filed in the name of Jordan, et al., published Mar. 28, 2007, the teachings of which are expressly incorporated herein by reference. Likewise exemplary of such teachings include those of U.S. Pat. No. 8,652,511 B2, entitled TRANSDERMAL DELIVERY PATCH, issued to Cottrell, et al. on Feb. 18, 2014, the teachings of which are expressly incorporated by reference. With respect to such reference, there is disclosed composition suitable for use in the transdermal delivery of both paclitaxel and docetaxel, and are believed to be suitable for use in the practice of the present invention. Still further exemplary teachings include those of Published United States Patent Application No. US 2005/0095283 A1, entitled COMPOSITIONS AND METHODS FOR TOPICALLY TREATING DISEASES, filed in the name of Castor, et al., published May 5, 2005; PCT Application No. PCT/US 2006/042827, entitled MACROCYCLIC FORMULATIONS FOR TRANSMEMBRANE DRUG DELIVERY, filed in the name of Gyunik, et al., published May 18, 2007, the teachings of which are likewise incorporated herein by reference.

In addition to such exemplary teachings, it will be understood and well within the competency of one of ordinary skill to use any of a variety of ingredients and formulations operative to derive a topically-administered pharmaceutical composition that achieves the objective of the present invention. Along those lines, well-known ingredients such as citric acid, polyethylene glycol, propylene glycol, stearyl alcohol, cocoa butter, cotton seed oil, olive oil, white petrolatum, water, mineral oil, methyl paraben, propyl paraben and other base materials as are frequently utilized as inactive ingredients in the formulation of creams, gels, foams, ointments, soaps, shampoos, lotions and the like, and can be selectively chosen based upon the compatibility with a particular CIA agent. Such ingredients are also readily available and can be incorporated in a variety of amounts as may be desired to formulate a particular pharmaceutical composition having a desired potency and ability for a particular CIA agent to become evenly and readily distributed over a specific surface area of skin such that an effective amount of a given CIA agent is sufficiently contacted with a given surface of skin to which the removal of hair is desired. Exemplary of such prior art teachings include those of Published United States Published Patent Application US 2011/0269704 A1, entitled METHOD FOR DEVELOPING A LIQUID COMPOSITION TO BE APPLIED TO THE SKIN AS A FOAM AND A COMPOSITION THAT CAN BE APPLIED TOPICALLY, filed in the name of Seigfried, published Nov. 3, 2011, the teachings of which are expressly incorporated herein by reference.

In addition to being able to formulate pharmaceutical compositions operative to deliver therapeutically effective amounts of the CIA agent to the localized area for which hair removal is desired, it will further be understood, that, when desired, other methodologies and/or compounds may be used to enhance the rate of absorption of the CIA agent so as to cause the CIA agent to penetrate the skin in order to produce the desired CIA effect. Accordingly, such pharmaceutical compositions may deploy additional components which act as an absorption promoter to facilitate transport of the CIA agent in sufficient amounts across the skin to achieve the desired effect. Among the types of known technologies operative to determine such formulations include those disclosed in PCT Patent Application No. PCT/US/2004/023634, entitled PENETRATION ENHANCER COMBINATIONS FOR TRANSDERMAL DELIVERY, filed by Mitragotri, et al., on Feb. 3, 2005, the teachings of which are expressly incorporated by reference. In addition, it is contemplated that other techniques, such as lightly abrading the skin and/or applying an electrical current, ultrasound or other enhancing forces to the skin so as to facilitate the ability of a particular agent to penetrate into the skin in amounts sufficient to produce a permanent CIA about the desired localized area. The general principles associated with the application of electrical fields and/or ultrasound to enhance transdermal drug delivery are set forth in U.S. Pat. No. 6,041,253 A, entitled EFFECT OF ELECTRIC FIELD AND ULTRASOUND FOR TRANSDERMAL DRUG DELIVERY, issued to Kost, et al., Mar. 21, 2000 the teachings of which are expressly incorporated herein by reference.

As will be appreciated by those skilled in the art, however, it is contemplated that through further experimentation and clinical testing will likely produce data indicative of optimal dosages and application regimens. Accordingly, it is contemplated that further refinements in dosing and applications may vary over time.

Along those lines, it is contemplated that the compositions of the present invention will be effective to effectuate permanent hair loss. However, to the extent hair regrowth does occur, it is contemplated that the compositions and methods of applying the same according to the present invention may be repeated as necessary so as to effectuate long term, preferably permanent hair removal upon discrete surface areas of the skin.

As discussed above, numerous other agents operative to produce permanent CIA when topically applied in an effective amount to an area of skin where selective alopecia is desired are deemed to fall within the scope of the present invention. Accordingly, in addition to taxanes and busulfan, other agents such as disulfan, cyclophosphamide, thiotepa, melphalan, etoposide, carboplatin, cisplatin, ifosfamide, doxorubicin, dactinomycin, vincristine, and combinations thereof may be formulated in a transdermally-applied composition such that the CIA agent is present in an amount effective to effectuate permanent hair loss upon a selectively discrete area of a user's body. As per the discussion above, it is contemplated that the agent will be formulated as part of a transdermally-applied composition that is formulated to be manually applied to a selective area of skin or otherwise formulated to be deployed through a transdermal drug delivery system, such as patches and the like.

As per the taxane embodiments discussed above, it is contemplated that the compositions may be formulated to address the hydrophobic or hydrophilic nature of the agents in question, or formulated to promote the stability of the drug, enhance the ability of the drug to become absorbed through the skin and ultimately be delivered in potent amounts effective to produce the desired CIA effect while also avoiding any type of systemic distribution throughout the body or beyond the desired site of application. Such considerations would be well-known and understood by those skilled in the art, and readily addressed using known pharmaceutical formulation techniques that is well within the knowledge of those of ordinary skill. For example, in compositions that utilize ifosfamide, it will be understood that any composition will be formulated to address its chemical properties, such as its solubility in water and its stability under certain conditions, including those as published in Trissel, L. A. Handbook on Injectable Drugs, 9^{th} ed. Bethesda, Md., American Society of Health-System Pharmacists' Product Development, 1996, p. 594, identifying the chemical and physical stability of ifosfamide at certain conditions and temperatures. Moreover, such composition will further necessarily take into account any activation typically required via microsomal liver enzymes to convert ifosfamide into its active metabolite in order to exert the desired cytotoxic effect. Again, such considerations are known within the art. Exemplary of such teachings include those of Published United States Patent Application No. US20140018435 A1, entitled TRANSDERMAL DELIVERY OF THERAPEUTIC AGENTS USING POLY(AMIDOAMINE) DENDRIMERS, published Jan. 16, 2014, the teachings of which are expressly incorporated by reference. Accordingly, it is believed to be readily understood by those skilled in the art that while the identification of a particular CIA agent for use in the present invention may be understood, it will further require taking into consideration the dosage amounts necessary to produce the desired CIA effect, developing a pharmaceutical composition operative to be topically applied and further operative to administer an effective amount of the CIA agent over a particular surface area of skin, and further enable the pharmaceutical composition to be stable, prevent to the highest degree possible any systemic distribution of the CIA agent, and further preferably formulate the product in such a way such that the CIA agent is absorbed through the skin in the safest and most efficient manner possible.

Additional modifications and improvements of the present invention may also be apparent to those of ordinary skill in the art. Thus, the particular combination of parts and steps described and illustrated herein is intended to represent only certain embodiments of the present invention, and is not intended to serve as limitations of alternative devices and methods within the spirit and scope of the invention.

What is claimed is:

1. A method for removing hair from skin comprising:
topically applying a composition upon an area of skin from which hair is sought to be removed, said composition containing an anti-cancer agent operative to effectuate chemotherapy-induced alopecia, said anti-cancer agent being present in an amount sufficient to effectuate chemotherapy-induced alopecia only upon said area of skin upon which said composition is topically applied.

2. The method of claim 1 wherein said composition is applied in a single application.

3. The method of claim 2 wherein said composition is applied via a transdermal drug delivery system.

4. The method of claim 1 wherein said anti-cancer agent for effectuating said chemotherapy-induced alopecia is a taxane.

5. The method of claim 4 wherein said taxane comprises docetaxel.

6. The method of claim 5 wherein said docetaxel and is applied in an amount ranging from 20 to 100,000 mg per square decimeter of skin.

7. The method of claim 6 wherein said docetaxel is applied in amount ranging from 200 to 10000 mg per square decimeter of skin.

8. The method of claim 7 wherein docetaxel is applied in an amount ranging from 200 to 2000 mg per decimeter of skin.

9-11.

12. The method of claim 1 wherein said composition is applied from 1 to 3 times per day for up to 365 days.

13. The method of claim 1 wherein said agent operative to effectuate chemotherapy-induced alopecia is docetaxel and is present in an amount ranging from 0.2 to 10 percent by weight of said composition.

14. The method of claim 13 wherein said agent operative to effectuate chemotherapy-induced alopecia is docetaxel and is present in an amount ranging from 0.2 to 2 percent by weight of said composition.

15-17.
